(19) United States
(12) Patent Application Publication
(10) Pub. No.: US 2011/0268666 A1
(43) Pub. Date: Nov. 3, 2011

(54) NOVEL GASTRORETENTIVE DELIVERY SYSTEM

(75) Inventors: Michael Friedman, Jerusalem (IL); David Kirmayer, Ma'ale Edomim (IL)

(73) Assignees: Yissum Research Development Company of the Research University of Jerusalem, Ltd.; Intec Pharma Ltd.

(21) Appl. No.: 13/121,546

(22) PCT Filed: Sep. 29, 2009
(86) PCT No.: PCT/IL09/00934

§ 371 Date: Jun. 9, 2011

Related U.S. Application Data
(60) Provisional application No. 61/136,723, filed on Sep. 29, 2008.

(51) Int. Cl. A61K 9/00; A61K 47/00; A61K 47/30; A61K 47/42; A61K 47/36; A61P 1/10; A61P 25/00; A61P 25/16; A61P 25/28; A61P 25/08; A61P 25/24; A61P 25/18; A61P 25/20; A61P 31/00; A61P 31/12; A61P 31/22; A61P 31/14; A61P 3/00; A61P 3/10; A61P 9/00; A61P 13/12; A61P 19/00; A61P 19/10; A61P 35/00; A61P 37/00; A61P 11/00; A61P 31/10; A61P 31/04; A61P 1/00; A61K 31/195

(52) U.S. Cl. 424/44; 514/561; 514/772; 514/772.3; 514/776; 514/777; 514/778; 514/781

(57) ABSTRACT

A novel gastroretentive delivery system comprising a tablet comprising a pharmaceutical ingredient or diagnostic, which tablet comprises a gas releasing ingredient or a tandem of two gas releasing ingredients, an ingredient capable of unrestricted swelling in gastric fluid, an ingredient capable of limiting the unrestricted swelling and a hardening ingredient. The said system is based on the use of three different gastroretentive mechanisms: flotation, swelling and mechanical strength, the three mechanisms acting in a complimentary way. Processes for manufacturing same and methods of treatment are also disclosed.

### CROSS-REFERENCE TO RELATED APPLICATIONS

The present application claims priority to U.S. Provisional Patent Application No. 61/136,723, filed on Sep. 29, 2008 and which is incorporated herein by reference in its entirety.

### FIELD OF THE INVENTION

The present invention relates to the field of pharmaceutics and more particularly, to a controlled-release drug delivery system for retention in the stomach for prolonged time periods of time using a novel technology combining three different mechanisms working in a synergistic or complementary way.

### BACKGROUND OF THE INVENTION

The advantages of using controlled-release drug delivery systems are many. Controlled drug therapy may reduce the required frequency of administration, ideally single doses at periodic intervals being sufficient, resulting in improved patient compliance. The main target is the improved efficiency in treatment.

However, conventional controlled-release drug delivery systems are of only limited use for (1) drugs having a “narrow absorption window” in the gastrointestinal tract, i.e. are preferentially absorbed in the duodenum and/or jejunum over ileum and/or colon; (2) local treatment of proximal parts of the gastrointestinal tract (stomach and/or duodenum); and (3) drugs which degrade in the colon. Controlled-release drug delivery systems intended for prolonged gastric retention in the stomach are known in the art. Several systems are described in the art, implementing various mechanisms. These include flotation on the surface of gastric content, sinking to the bottom of the stomach, unfolding dosage forms, bioadhesive dosage forms, swelling or expandable dosage forms, and various delivery systems, comprising more than one of the above mechanisms. The controlled-release drug delivery systems for gastric retention were extensively reviewed lately (AAPS Pharm. Sci. Tech. 2005; 6 (3) Article 47-Sh. Arora et al, Floating Drug Delivery Systems: A Review), Journal of Controlled Release, 63 (2000) 235-259, “Floating drug delivery systems: an approach to oral controlled drug delivery via gastric retention”; J Control. Release, 2003; 90 (2): 143-62, “Expandable gastroretentive dosage forms”. Klausner E. A., Lavy E, Friedman M, Hoffman A.; Exp. Opin. Drug. Deliv. 2006; 3 (2): 217-33, “Gastroretentive drug delivery systems”. Streubel A, Siepmann J, Bodmeier R.

There are several drawbacks in each of the individual presented above mechanisms of action, which unfortunately limit their use in modern pharmaceutics. Floating drug delivery systems were shown to be heavily dependent on the volume of stomach's content, and to be dependent on postural changes of the patient. They also suffer from binary clearance from the stomach, meaning once the dosage form is cleared, it can no longer serve as a platform for drug delivery. The latter drawback has been addressed using multiparticulate floating delivery systems, which are not cleared as one unit from the stomach, and possess the advantage of continuous efficacy even under conditions wherein the initial load of floating particles, sometimes referred to as “microballoon”, is partially cleared from the stomach of the patient. Bioadhesion, on the other hand, has solid theoretical grounds for efficacy, which is however limited by the rapid turnover of the gastric mucus whereto the bioadhesive systems adhere, and the need for applied pressure in order to initiate the bioadhesion. Another shortfall of the bioadhesive dosage forms is the irritant nature of the adhesion to gastric mucosa, if performed, and gastric erosions development as the dosage forms thus applied facilitate the contact between the gastric wall and the gastric milieu. High-density dosage forms were contemplated some decades ago, and were abandoned due to lack of efficacy.

Swelling tablets have been coveted for years by the pharmaceutical industry and much research has been invested to obtain a tablet capable of swelling to large dimensions. Superporous hydrogel composites, capable of almost instantaneous swelling to equilibrium, were recently reviewed, including possible application to pharmaceutical technology (J. Pharm. Pharmacol. 2007; Mar 59(3): 317-327; “Recent developments in superporous hydrogels”, Omidan et al). Other swelling systems can be exemplified by the work described in U.S. Pat. No. 6,723,340 (to Depomed). The limitations of the swelling tablets are exemplified by the work of the same group in U.S. Pat. No. 6,635,280, “Extending the duration of drug release within the stomach during the fed mode”, that discusses the advantages of obtaining gastric retention under high-calorie meal. Under other conditions, as S. Davis states in Drug Discovery Today, Vol. 10, Issue 4, Feb. 15 2005, 249-257, “ . . . . Tablets formulated using hydrophilic polymers were tested in dogs and were found to have excellent gastroretentive properties (greater than 12 h) even in the fasted state. Sadly, when these systems were tested in human volunteers using scintigraphy, the average time for emptying from the fasted stomach was just 33 minutes . . . ”.

Relatively large dosage forms are needed to ensure gastric retention. It has been shown in the unfolding system described in U.S. Pat. No. 4,758,436 (to Merck and Co), that dimensions of at least 1.6 cm to maximum of 5.0 cm are needed, while the dosage form is also sufficiently resilient to simultaneous contractions in two directions. Also, the need for a shape that would allow easy swallowing for large dosage forms is considered a pre-requisite for any gastroretentive tablet. For example, U.S. Pat. No. 4,140,755 (to Hoffman—La Roche Inc) describes a “ . . . capsule-shaped tablet with dimensions ¾″× 5/16″ (1.9×0.8 mm) (column 10, lines 1-2) . . . ”. Along the same lines, WO 01/97783 A1 (to DepoMed) claims the shape of a tablet for gastric retention of a particular orientation, with longer axis of projection being short enough to allow easy swallowing (30 mm) and the shorter one reaching at least 12 mm after 1 hour of being immersed into water.

### SUMMARY OF THE INVENTION

The gastric retention system of the present invention is based on the activation of three different mechanisms in the system of the present invention:

### 1. Flotation

### 2. Swelling

3. Hardening, or retention of initial hardness in the swollen state

The three above mechanisms act in concerted or complementary way and in synergy with each other, but they do not need to be simultaneously activated on the timeline.

The present invention provides a system in the form of a tablet, for retention in the stomach for prolonged time periods. The gastric retention effect is provided by the activation of the three above mechanisms in a concerted or complementary way. Thus, the three mechanisms work in a concerted or complementary way by compensating for each other, so that the requirements from each one of them are less stringent. The activation of the three mechanisms on the timeline, their onset of action and their specific parameters are modulated by changes in the formulation of the gastroretentive system of this invention.

A preferred mechanism of retention is as follows:

a. a rapid onset of floating, within 5 to 15 minutes;
b. gradual swelling to final dimensions, not less than within 30 to 60 minutes;
c. development or retention of mechanical strength in the swollen state.

Despite the above, all three mechanisms overlap in some measure on the timeline.

The main trait of the present invention is the principle of mutually-entangled mechanisms, inseparable from each other, to obtain gastric retention, as a concept of “Flotation/Swelling/Hardness”. The system has to become afloat upon contact with gastric fluid as rapidly as possible, and it floats as long as the system swells to its final dimensions, whereupon the flotation is not essential anymore though preferable. The system develops substantial mechanical strength while floating and not after the flotation is over, and it is characterised by relatively high values of mechanical properties, as measured by suitable tests and described in more detail hereinbelow. The system also increases its dimensions considerably, but only to a size that can impede the passage through pyloric sphincter, whereas the swelling occurs while the tablet floats. The system does not need to employ unrestricted swelling to perform satisfactorily.

### BRIEF DESCRIPTION OF THE DRAWINGS

FIG. 1 shows the stress-strain curves at various ratios of hypermelose: ovalbumin blends in the system of this invention, according to example 3.

FIG. 2 shows the various degrees of swelling of the sample tablets at various pH values, according to example 4.

FIG. 3 provides the stress-strain curves of the sample tablets of this invention, swollen at various pH values, as measured by compression test, according to example 4.

FIG. 4 provides the gabapentin release over time and the standard deviation thereof, according to example 5

### DETAILED DESCRIPTION OF THE INVENTION

### Definitions

The wording hereinbelow is implied in the common meaning of the definitions and statements as known to the versed in the art of pharmaceuticals and polymer science. However, there are several terms that should be understood in the concept of present invention as follows:

“Gastroretentive”, “gastric-retentive” and the like are implied with reference to objects being capable of residence in the stomach of a mammal, preferably a human, for prolonged periods of time, preferably longer than that of food and small indigestible particles of size below 10 mm in either dimension. “Gastric retention” is therefore the maintenance or withholding of a drug in stomach, for a time period longer than the time it would have been retained in the stomach when delivered in a free form or within a gastro-intestinal (GI) delivery vehicle which is not considered gastroretentive. Gastroretentivity may be characterized by retention in the stomach for a period that is longer than the normal emptying time from the stomach, i.e. longer than about 2 hours, particularly longer than about 3 hours and usually more than about 4, 6, 8 or 10 hours. Gastroretentivity typically means retention in the stomach from about 3, 4, 6, 8, 10 or at times 18 hours up to about 21 hours. It is however noted that in accordance with the invention, retention of the controlled-release drug delivery system is not observed after more than 48 hours after administration to non-fasting stomach, and preferably not after 24 hours.

“Controlled-release drug delivery system” is used herein in reference to a dosage form of an active pharmaceutical ingredient, whereas the latter is released from the said dosage form in a controlled manner over designable time intervals at needed quantities to produce a prolonged, sustained or delayed pharmacological effect that is otherwise unattainable through conventional non-modified-release dosage forms. More specifically, the term is referring to the system of present invention. It can also be generally understood as known to the versed in the art.

“Swelling” is used herein in reference to the ability of an ingredient to increase its dimensions, usually upon contact with a medium wherein it is capable of swelling through increasing the free volume between the molecules of the ingredient or between the strands of a polymer by a process whereby the molecules of the swelling medium are incorporated into the said free volume, therefore increasing the total volume of the system. When referred to the whole system of the invention, swelling means the capability of the system to increase its dimensions beyond the initial size of the system. “Unrestricted swelling” is implied in connection with the present invention as unimpeded swelling of the ingredient according to the definition above, such swelling as would occur if the ingredient were subjected to the unlimited amount of swelling medium and were left therein for sufficient time for the ingredient to reach equilibrium whereby the amount of swelling medium penetrating the ingredient is equal to amount of the said medium leaving the ingredient. “Restricted swelling” refers herein to a swelling whereby the maximal swelling that would be obtained in case of unrestricted swelling is not achieved, whereas the factors impeding the unrestricted swelling are the ingredients and processing methods of the system of present invention “Swelling” can be characterised by increasing the dimensions of the system of present invention to a size over 10 mm in either direction, particularly more than 12 mm and preferably more than 16, 20 and 24 mm in at least one direction, and at least 140% increase of the initial projection area of the swollen system to a planar surface, relatively to a dry tablet. Most preferably, “swelling” is characterised by increasing the dimensions of the initial tablet to the size that would not readily be cleared from the stomach. Ideally but not necessarily, “swelling” is characterised by increasing the dimensions over 20 mm in at least two directions, but possibly only in one direction, with the other being substantially large, being at least 30% of the larger dimension, preferably over 35, 40 and 50% of the larger dimension.

“Floating”, “flotation” and “buoyancy”, used interchangeably, are used herein in reference to the ability to position the system of the invention onto or in the proximity of the surface of the gastric medium.

“Hardening”, “developing mechanical strength” or “retaining mechanical strength” are used in connection with the present invention in reference to the ability of the system to attain such mechanical properties as to enable structural integrity throughout the time period that the system performs according to the design. In particular, the mechanical properties imply considerable mechanical strength in comparison to swollen gel tablets, such strength being higher than 300 g required to deflect the swollen gel by 4 mm.” For such ability to attain said mechanical properties the direction of change of such properties is immaterial, whereas the terms “hardening” and “developing mechanical strength” refer to the described ability from weaker starting points, and the term “retaining” refers to the ability of never reaching weaker points than the end-point of the mechanical strength.

“Treating” or “treatment”, and the like are used herein to refer to obtaining a desired pharmacological and physiological effect. The effect may be prophylactic in terms of preventing or partially preventing a disease, symptom or pathological condition and/or may be therapeutic in terms of a partial or complete cure of a disease, condition, symptom or adverse effect attributed to a pathological condition. Thus, “treatment” covers any treatment of a disease in a mammal, particularly a human, and includes: (a) preventing a pathological condition from occurring in an individual which may be predisposed to develop a pathological condition but has not yet been diagnosed as having it, i.e., causing the clinical symptoms of a pathological condition not to develop in a subject that may be predisposed to develop the condition but does not yet experience or display symptoms of the condition; (b) inhibiting, i.e., arresting or reducing the development of the pathological condition or its clinical symptoms; or (c) relieving symptoms associating with the pathological condition.

“Pathological condition” used herein denotes any condition which requires for improving the well-being of the subject by the delivery of a biologically functional and active drug, the latter being as defined herein. This includes, inter alia, a condition selected from inflammation and autoimmune disorders, parasitism (e.g. malaria), bacterial, viral or fungal infection, cardiac disorders (e.g. arrhythmia), coagulation disorders, depression, diabetics, epilepsy, migraine, cancer, immune disorders, hormonal disorders, psychiatric conditions, gastrointestinal tract disorders, nutritional disorders, and many others, as known in the art.

“Condition of the GI tract” (used interchangeably with the term “GI pathological condition”) denotes any condition of the GI tract, preferably the stomach or the small intestine, which is associated with an abnormality of the GI tract. This includes a disorder or disease where the primary abnormality of the GI tract is an altered physiological function (the way the body works) such as in the case of irritable bowel syndrome (IBS) and dyspepsia (which are the most common functional GI disorders), as well as structural disorders (having an identifiable structural or biochemical cause, such as in the case of GI polyps, cancer, ulcer etc.).

“Drug”, “active pharmaceutical ingredient”, API, “active agent”, “active ingredient” “active”, and the like, are used in connection with the present invention as pure chemical substances, mixtures of pure chemical substances or crude extracts from various sources, which are used to treat pathological conditions of a person in need or such treatment. Typically, drugs are active pharmaceutical ingredients (APIs), known in pharmacotherapy of various disorders, and normally an API is deemed as a pure chemical substance, either synthetic or semi-synthetic, whereas its exact chemical structure is known and pharmacodynamic profile is well established or not. This term is aimed to embrace such APIs, existing to the date of writing, without excluding, however, the APIs that can be used in pharmacotherapy in general. Therefore, whenever the wording refers to a drug, it refers both to any existing drug and to a newer drug, as would be appreciated by a skilled in the art at relevant times.

“Inactive ingredient”, “excipient”, “material”, “component”, “ingredient”, “inactive material”, “inactive”, “agent”, and the like as used interchangeably herein, refer to materials or ingredients that are not drugs, but are employed in pharmaceutical compounding in connection with the present invention with intention to impart the final dosage form specific characteristics, as known to the versed in the art, and partially described in detail hereinbelow with reference to specific characteristics imparted to the system of present invention.

As used in the specification and claims, the forms “a”, “an” and “the” include singular as well as plural references unless the context clearly dictates otherwise. For example, the term “an agent” includes one or more, of the same or different agents.

Further, as used herein, the term “comprising” is intended to mean that the system includes the recited elements, but not excluding others which may be optional in the design of the system, such as fillers and the like. The term “consisting essentially of” is used to define a system that includes the recited elements but exclude other elements that may have an essential significance effect on the performance of the system. “Consisting of” shall thus mean excluding more than traces of other elements. Embodiments defined by each of these transition terms are within the scope of this invention.

The tablet of this invention is retained in the stomach due to the synergistic combination of three different synergistic retention mechanisms working in concert or complementing each other.

It is an embodiment of this invention to provide a novel gastroretentive drug delivery system in the form of tablet, capable of retention in a mammal (preferably a human) stomach wherein said system comprises at least three different gastroretentive mechanisms, working in concert or complementing each other.

In another embodiment, the above three different gastroretentive mechanisms are:

### 1. Flotation

### 2. Swelling

3. Hardening, or retention of the initial hardness in the swollen state

In yet another embodiment, the three above mechanisms act in synergy.

While the three above mechanisms act in concert, complementary and in synergy with each other, they do not need to be simultaneously activated on the timeline.

The present invention provides a system in the form of a tablet intended for swallowing, for retention in the stomach for prolonged time periods. The gastric retention effect is provided by the activation of the three above mechanisms in a concerted or complementary way.

The three mechanisms work in a concerted or complementary way by compensating for each other, so that the requirements from each one of them are less stringent. The activation of the three mechanisms on the timeline, their onset of action and their specific parameters are modulated by changes in the formulation of the gastroretentive system of this invention.

A preferred mechanism of retention is as follows:

a. a rapid onset of floating, within 5 to 15 minutes;
b. gradual swelling to final dimensions, not less than within 30 to 60 minutes;
c. development or retention of mechanical strength in the swollen state.

Despite the above, all three mechanisms overlap in some measure on the timeline.

The gastroretentive drug delivery system of this invention becomes buoyant 5 to 30 minutes upon contact with gastric fluid.

The swelling to final dimensions of the gastroretentive system of this invention takes place not earlier than within 30 to 60 minutes.

The main trait of the present invention is the principle of mutually-entangled mechanisms, inseparable from each other, to obtain gastric retention, as a concept of “Flotation/Swelling/Hardness”. The system has to become afloat upon contact with gastric fluid as rapidly as possible, and it floats as long as the system swells to its final dimensions, whereupon the flotation is not essential anymore though preferable. The system develops substantial mechanical strength while floating and not after the flotation is over, and it is characterised by relatively high values of mechanical properties, as measured by suitable tests and described in more detail hereinbelow. The system also increases its dimensions considerably, but only to a size that can impede the passage through pyloric sphincter in either orientation, whereas the swelling occurs while the tablet floats. The system does not need to employ unrestricted swelling to satisfactorily perform.

The tablet may develop or retain its mechanical strength in the swollen state.

These three mechanisms act in a synergistic and complementary way, compensating for each other. While the three mechanisms coexist and act in concert, complementing each other, they may predominate in one stage or the other of the gastric retention period.

Thus, as the three mechanisms work in a concerted or complementary way by compensating for each other, the requirements from each one of them are less stringent. The activation of the three mechanisms on the timeline, their onset of action and their specific parameters are modulated by changes in the formulation of the gastroretentive system of this invention

Without wishing to be bound by a specific theory, we believe the rationale for the preferred order of action is as follows: rapid onset of flotation provides a primary mechanism of gastric retention in the early stages of functioning of the system; thereby the tablet is kept far apart from the pyloric sphincter, enabling the swelling ingredients of the tablet to be activated, not in a burst-like manner, but in a rather sustained, more natural way of hydrogel swelling. This slow swelling allows for the fine kinetic tuning of hardening of the tablet through a physical or a chemical process in case of de-novo development of mechanical strength, or for ensuring retention of mechanical strength and shape in cases where initially hard systems are used.

While there is a preference for a specific order of onset of the three mechanisms, their synergy and complementary and compensatory action allows unusual latitude, as detailed in the following.

The synergy of the three above mechanisms makes the rapid swelling, which is necessary in some other gastroretentive systems, not a pre-requisite in the system of the present invention. While recognising the positive influence that swelling to unrestricted size may have on the functioning of the system, in the disclosed invention the swelling is intended only to a size that would impede easy expulsion from a stomach, such as to the sizes comparable with the pyloric valve in an open state, as the system floats and, having developed the intended mechanical strength, does not erode under the peristaltic movement of the stomach. The instantaneous maximal swelling, which is always desirable for regular swelling systems, dependent mainly on swelling as a mechanism of gastric retention is therefore not a pre-requisite for the system of the present invention. In one of the embodiments, the gastroretentive drug delivery system of this invention becomes buoyant upon contact with gastric fluid, swells to final dimensions and is hard in the swollen state, in this order of action.

In another embodiment, the present invention does not need flotation to be maintained throughout the course of performance of the system, unlike other gastric retentive delivery systems in the field. While recognising the positive influence that permanent flotation may have on the functioning of the system, the flotation in the present invention is intended as only one of the mechanisms to prevent premature clearance from the stomach, before the system swells to final dimensions or develops the intended mechanical strength.

In the system of the present invention, unlike other gastric retentive systems, the extensive unrestricted swelling is not a pre-requisite, as strength and flotation compensate the performance. On the contrary, restricted swelling is advantageously employed to yield a controllable mechanical strength of the swollen system, which is otherwise hard to achieve. There is also no need for rapid swelling kinetics, as other mechanisms cover initial periods. In preferred embodiments, swelling can be characterised as set forth in definitions section, by increasing the dimensions of the system of present invention to a size over 10 mm in either direction, particularly more than 12 mm and preferably more than 16, 20 and 24 mm in at least one direction, and at least 140% increase of the initial projection area of the swollen system to a planar surface, relatively to the dry tablet. In further preferred embodiments, swelling increases the dimensions of the initial tablet to the size that would not readily be cleared from the stomach.

Summing up, the synergy and/or the compensatory and complementary action of the three mechanisms in the novel gastroretentive system of this invention provides unique latitude previously unattained in the previously known controlled-release drug delivery systems intended for prolonged gastric retention in the stomach.

The gastroretentive drug delivery system of this invention performs best when all three said mechanisms occur during the same performance run.

In a preferred embodiment, the gastroretentive drug delivery system of this invention comprises a tablet suitable for swallowing by a patient, which tablet comprises a therapeutically effective amount of at least one active pharmaceutical ingredient or diagnostic, an inactive ingredient capable of releasing gas upon contact with gastric fluids or a tandem of gas-generating ingredients or an ingredient producing a low-density phase, an inactive ingredient capable of unrestricted swelling in gastric fluid, an inactive ingredient capable of limiting the unrestricted swelling of the said swelling ingredient and an inactive hardening ingredient which allows for the formation of mechanical strength or retention thereof by any mechanism, wherein said inactive ingredients are capable of performing more than one of the functions above.

In another preferred embodiment, the above gastroretentive drug delivery system of this invention comprises as unrestricted swelling ingredient egg albumin or the swelling-restricting ingredient therein is denaturated egg albumin or both.

In another preferred embodiment, the gastroretentive drug delivery system of this invention comprises at least one ingredient capable of releasing gas upon contact with gastric fluids, at least one ingredient capable of unrestricted swelling in gastric fluid, at least one ingredient capable of limiting the unrestricted swelling of the said swelling ingredient, and at least one therapeutically active drug, or any combination of ingredients capable of performing more than one of any of the functions above, and the said gas releasing ingredient is a physical mixture of gas-releasing basic salt and an organic or inorganic acid.

In another embodiment, the gas released by the gas-releasing mixture enables flotation of the gastroretentive drug delivery system of this invention.

In one embodiment, the gas-releasing mixture above may comprise a bicarbonate.

In another embodiment, the gas-releasing mixture comprises citric acid, tartaric acid, or a mixture thereof.

In yet another embodiment, the gas-releasing mixture further comprises a polymer.

In a further embodiment, the above polymer further comprises a plasticizer.

In one of the embodiments, the gastroretentive drug delivery system of this invention comprises at least one ingredient capable of releasing gas upon contact with gastric fluids, at least one ingredient, capable of unrestricted swelling in gastric fluid, at least one ingredient capable of limiting the unrestricted swelling of the said swelling ingredient, and a therapeutically effective amount of at least one drug, or any combination of ingredients, capable of performing more than one of any of the functions above, and the said swelling ingredient is a hydrogel, a protein, a water-soluble resin, a gum or an insoluble swelling ingredient.

In another embodiment, the gastroretentive drug delivery system, wherein the swelling ingredient is chosen from hypromelose, methyl cellulose, hydroxypropyl cellulose, hydroxyethyl cellulose, carboxymethyl cellulose, guar gum, xanthan gum, locust bean gum, tragacanth gum, carrageenan, pectin, dextrins, egg albumin, bovine serum albumin, casein, gelatine, zein, polyvinyl alcohol, graft polyvinyl alcohol-polyethylene glycol, polyethylene oxides, microcrystalline cellulose, cross-linked carboxymethyl cellulose, cross-linked polyvinyl pyrrolidone, polacrylin, or a starch.

In a further embodiment, the above swelling ingredient is egg albumin.

In one of the embodiments, the gastroretentive drug delivery system of this invention comprises at least one therapeutically active drug, at least one ingredient capable of releasing gas upon contact with gastric fluids, at least one ingredient capable of unrestricted swelling un gastric fluid, at least one ingredient capable of limiting the unrestricted swelling of the said swelling ingredient, or any combination of ingredients capable of performing more than one of any of the functions above, and the said swelling-restricting ingredient is a small molecule.

In another embodiment, the small molecule is chosen from tannic acid, glutaraldehyde and formaldehyde.

In a further embodiment, the gastroretentive drug delivery system of this invention comprises at least one ingredient, capable of releasing gas upon contact with gastric fluids, at least one ingredient capable of unrestricted swelling un gastric fluid, at least one ingredient, capable of limiting the unrestricted swelling of the said swelling ingredient, and a therapeutically effective amount of at least one drug, or any combination of ingredients, capable of performing more than one of any of the functions above, and the said swelling-restricting ingredient is a polymer.

In another embodiment, the polymer is an elastic polymer, capable of substantial elongation under stress. The elastic polymer can further comprise a plasticizer.

In a further embodiment, the polymer is a synthetic polymer, wherein the polymer is chosen from polymethacrylates, ethyl cellulose, hypromelose phthalate, hydroxypropyl methyl cellulose acetate succinate, cellulose acetate phthalate, cellulose acetate, cellulose acetate butyrate and other polymers.

In another embodiment, the polymer further comprises a plasticizer, said suitable plasticiser, is chosen from polyethylene glycols, poloxamer block copolymers, citrates, such as triethyl citrate or tributyl citrate, phthalates, such as diethyl phthalate, dibutyl sebacate, mineral oil, triglycerides, fatty acids, fatty acids alcohols, such as cetyl alcohol, and others.

In a further embodiment, the polymer is cross-linked egg albumin.

In another embodiment, the cross-linked egg albumin is produced through controlled denaturation of native egg albumin powder.

In yet another embodiment, the controlled denaturation of native egg albumin powder is instigated by an organic solvent. The organic solvent may be added during the granulation step.

In a further embodiment, the organic solvent is isopropanol.

It is understood that one ingredient may serve as both a swelling and as strength retention ingredient or any other combination of the roles listed above. The ingredients can be selected according to their suitability to the system, as known to those versed in the art, and examples of such ingredients can be found, for instance, in the IIG (Inactive Ingredients Guide) or in the GRAS (Generally Recognized As Safe) lists of the Food and Drug Administration of the USA.

In preferred embodiments, the tablet comprises at least one active pharmaceutical or diagnostic compound, at least one ingredient capable of development of a gas, or ingredient producing a low-density phase, at least one ingredient capable of swelling in gastric medium and at least one ingredient, capable of restricting the swelling of the swelling ingredient, wherein the swelling restricting ingredient is a polymer, a plasticised polymer, a polymer blend or any other combination of one or more polymers and one or more small molecules.

In another preferred embodiment, the tablet comprises at least one active pharmaceutical or diagnostic compound, at least one ingredient, capable of development of a gas or ingredient producing low-density gels, at least one ingredient, capable of swelling in gastric medium and at least one ingredient capable of restricting the swelling of the swelling ingredient, wherein the swelling restricting ingredient is a cross-linking compound, either a small molecule or a polymer, or a ingredient, capable of releasing of a suitable cross-linking compound at the site of action, in gastric medium.

In yet another preferred embodiment, the swelling-restricting compound comprises a chemically or physically cross-linked swelling compound, and in some instances, such compound is cross-linked on the surface of the swelling compound.

The gastroretentive drug delivery system of this invention when swollen upon the contact with gastric fluid possesses substantially hard mechanical characteristics, capable of resilience to compressive forces of the stomach.

In a preferred embodiment, the said swelling-restricting ingredient may be capable of swelling by itself if subjected to aqueous environment, or alternatively it is capable of elastic elongation, complying with the swelling of the swelling ingredient to some extent, and limiting the swelling to develop mechanical strength, wherein the swelling-restricting ingredient is not a cross-linking agent.

In some preferred embodiments, the examples of such ingredients suitable for performance as swelling-restricting ingredients, are proteins exemplified by egg albumin, milk casein, gelatine and its derivatives, and others. Preferably, the proteins are partially or fully denaturated, or partially or fully cross-linked. Another example of such ingredients is synthetic polymers, especially the ones that are in use in compounding pharmaceutical dosage forms. These include polymethacrylates, as defined in the United States Pharmacopeia, cellulose derivatives, such as ethyl cellulose, hypromelose phthalate, hydroxypropyl methyl cellulose acetate succinate, cellulose acetate phthalate, cellulose acetate, cellulose acetate butyrate and other polymers. Optionally, the swelling-restricting ingredient comprises a suitable plasticiser, as known to those versed in the art. Examples of plasticisers, suitable in connection to the citrates, such as triethyl citrate or tributyl citrate, phthalates, such as diethyl phthalate, dibutyl sebacate, mineral oil, triglycerides, fatty acids, fatty acids alcohols, such as cetyl alcohol, and others.

The preferred swelling-restricting ingredient is an at least partially denaturated ovalbumin (egg albumin).

Swelling within the system is achieved by the means of a swelling ingredient. In preferred embodiments the swelling ingredient should possess the quality of being capable of interaction with other ingredients or with itself (intra- or inter-molecularly) to preserve mechanical strength in the swollen wet state or to build de novo the properties of substantial mechanical strength. In some embodiments such interaction is a chemical or physical cross-linking of the polymeric strands. In further embodiments the cross-linking agent is an active group on the side chain of the swelling ingredient, an ion, a small polyvalent molecule, or a polymer. The swelling ingredients are known to those versed in the art, and these comprise cellulose hydrogels, gums, proteins, water-soluble resins and swelling insoluble ingredients. Hydrogels can be exemplified by cellulose derivatives, such as hypromelose, methyl cellulose, hydroxypropyl cellulose, hydroxyethyl cellulose, carboxymethyl cellulose and others. Gums are resinous ingredients that swell in aqueous media, and can be exemplified by guar gum, xanthan gum, locust bean gum, tragacanth gum, carrageenan, pectin, dextrins, and other related ingredients. Proteins can be exemplified by egg albumin, bovine serum albumin, casein, gelatine, zein and other ingredients. Water-soluble resins in the connexion to the invention can be exemplified by polyvinyl alcohol, graft polyvinyl alcohol-polyethylene glycol, polyethylene oxides and povidones. Water-insoluble particles can comprise microcrystalline cellulose, cross-linked carboxymethyl cellulose, cross-linked polyvinyl pyrrolidone, known as crosspovidone, polacrylin resins and other ingredients. Another suitable ingredient can be chosen from the group of starches.

The preferred swelling ingredients are egg albumin, hypromelose and mixtures thereof.

In another embodiment, the system of the present invention uses a protein as a swelling ingredient. Proteins possess several potential cross-linkable groups, therefore enabling “reinforceability”, and they claim the advantage of elastic matrix former. One major advantage of proteins is their denaturation under relatively mild conditions. Denaturation can be accompanied by various processes, including inter- and intra-molecular cross-link formation, thereby providing a system wherein both swelling and swelling-restriction ingredients are formed simultaneously.

Both swelling and swelling-restricting ingredients can comprise ingredients capable of mechanical reinforcing of polymers. For example, particle reinforcement of polymers has been known for decades (automobile tires comprising carbon-reinforced rubber), but have found limited implementation in pharmaceutical sciences. Thus, small inorganic or organic particles can be incorporated into the swelling and swelling-restricting ingredients. Examples of pharmaceutically acceptable inorganic particles are colloidal silicon dioxide, talc, bentonite (known in materials science as montmorellonite), kaolin, calcium carbonate and others.

In yet another embodiment, the flotation of the system of the present invention is achieved using gas-generating ingredients, such as sulphites, bicarbonates, coupled with a suitable organic or inorganic acid in what is termed a tandem gas-generating system. The acid is either an endogenous fluid, such as gastric secretions, or is an externally added pharmaceutical excipient, such as citric acid, tartaric acid or a suitable mixture of the same.

In a preferred embodiment, the flotation is achieved using a gas-generating tandem of organic acid and carbon-dioxide releasing ingredient. In further preferred embodiments the gas-generating substance is sodium bicarbonate. In one embodiment, the acid is an endogenous fluid, such as gastric fluid. In another preferred embodiment, the organic acid is added to the tablet's formulation. In a further embodiment, the added acid is citric acid, tartaric acid, or a mixture thereof.

In one embodiment, the gas-generating system can also comprise entrapping excipients, to enhance the gas retention within the delivery system and improve the flotation. For this purpose, several polymers are suitable. Thus, in one embodiment, the entrapping polymer is a hydro gel, such as hypromelose, hydroxypropyl cellulose, alginic acid and similar ingredients, described above in the connection with swelling ingredients. In another embodiment, the entrapping polymer is a hydrophobic polymer. Such polymers can be exemplified by pharmaceutical polymethacrylates, optionally comprising a preferably hydrophobic plasticizer, exemplified by diethyl phthalate, dibutyl sebacate, castor oil derivatives and other lipids. In preferred embodiments, the entrapping polymer is a hydrogel, since hydrogel can also contribute to another important parameter of the system, such as swelling.

In another preferred embodiment of this invention, there is provided a gastric retentive delivery system that uses three different mechanisms to improve the gastric retention and thus provide improved therapeutic action, wherein the simultaneous and continuous activation of all three mechanisms on the timeline is not a pre-requisite in providing an adequate gastric retention period.

In another embodiment, the system of the present invention claims the use of egg albumin, known also as ovalbumin, egg albumen and under other names, as the swelling carrier protein. Other proteins are suitable for the system include bovine albumin, casein, collagen and other food proteins, which can be used either as purified ingredients or as crude food-grade products.

In yet another embodiment, the system of the present invention also claims the advantage of controlled denaturation of ovalbumin, using various methods. Denaturation methods of ovalbumin in solution under heat, of dry powder under heat and in presence of glycerine, of dry powder under heat and in presence of moisture were described in the literature. The preferred denaturation method of ovalbumin with reference to the present invention is organic solvent denaturation, either under heat or at ambient temperature. We believe that this type of denaturation is brought about by the partial disruption of van der Waals forces of the egg albumin strands and subsequent oxidation of cysteine residues thereon. There are, however, organic solvents that do not readily yield denaturation of ovalbumin, so this denaturation process is not a trivial one. However, isopropanol proved suitable, therefore, the preferred denaturation organic solvent is isopropanol.

In several preferred embodiments, the system of this invention also contains surface-active materials or ingredients (SAMs). It was shown in several studies that disruption of hydrophobic interactions through SAMs, used in testing medium, improves the swelling properties of ovalbumin. SAMs can be added to the formulation either at the granulation step, or at the final blending step. Preferably, the SAM is incorporated at the granulation step. SAMs can be non-ionic, zwitterionic, cationic or anionic. The preferred SAMs are sodium lauryl sulphate, as anionic SAM example, or polysorbate 80, as a non-ionic SAM example.

In one of the embodiments, the surface-active material (SAM) is selected from a cationic SAM, an anionic SAM, a zwitterionic SAM or a non-ionic SAM.

In a further embodiment, the said surface-active material is selected from polysorbates, poloxamers, sodium alkyl sulphonates, lecithin, cholic acids and other related ingredients.

In another embodiment, the system comprises disintegrants. The use of disintegrant for enhanced water imbibition has also been reported in several sources quoted above. The disintegrants possess wicking properties, which are employed in their direct action, whenever disintegrant is used in appropriate amount. The amount of disintegrant in a formulation is sufficient if no gelling occurs within those inactive ingredients, which comprise cross-linked polymers. If a disintegrant is employed beyond the disintegration limit, it is capable of forming a gel that is rapidly wetted. This function is successfully exploited in wetting of hydrogels, which are only slowly wetted if at all, without erosion. Therefore, disintegrants are valuable excipients in relation to the present invention. The disintegrants can be applied inside the granulation powder, termed intragranularly, or can be added to the blending step, termed extragranularly, acting preferentially as reinforcement agents or as a wetting agent, respectively. In connection with the present invention, disintegrants are used both to improve water penetration and to strengthen the ovalbumin matrix. While the mechanism of such strengthening effect is not obvious, we believe that intragranular disintegrants act as swelling ingredients, whereas at least partially denaturated albumin acts as swelling restricting ingredient, or through a specific interaction with albumin strands' moieties.

In another embodiment, the system of this invention can further comprise a pH modulating agent inside the matrix, to control the degree of neutralisation of ovalbumin. As ovalbumin comprises carboxylic acid residues, partial or complete neutralisation can serve as basis for changes in basic physicochemical properties of ovalbumin, for example, improvement of water uptake by the system. The agent can be added to cause pre-neutralisation of the parent, non-neutralised ovalbumin, or alternatively, can be added to cause neutralisation during the performance of the system in vivo or in vitro.

In another embodiment, the system can comprise an additional swelling ingredient. This additional swelling ingredient is not limited by any physicochemical property, unlike the embodiments wherein the swelling agent also performs as swelling-restricting ingredient. This swelling agent can further be a part of gas-generating system as a gas-entrapping excipient, or a stand-alone ingredient, aimed at increasing the swelling of the system. The examples for such ingredients are listed above in the section, dealing with swelling ingredients.

In yet another embodiment, the system comprises an additional wetting ingredient. The additional wetting ingredient augments the water intake by the system, enabling timely swelling of the system. The examples of such ingredients are liquid wetting ingredients such as glycerine, polyethylene glycol, and others; and solids, exemplified by either a soluble solid, such as soluble fillers, like lactose, mannitol, sucrose, povidone, or insoluble ingredients, such as microcrystalline cellulose, disintegrants, and other ingredients.

It must be understood that in order to produce such a system, the swelling-restricting ingredient must at least partially enrobe the swelling ingredient. This is achieved naturally when the swelling-restricting ingredient is a cross-linked version of the swelling ingredient, more particularly surface-cross-linked version of a swelling ingredient. In other embodiments, the relative particle size of the ingredient is critical to formation of this system. Therefore, in some embodiments, the particle size of swelling-restricting ingredient is substantially smaller than that of the swelling ingredient, in order to enable the formation of the matrix.

Another embodiment of this invention provides systems, wherein the swelling-restricting agent acts in a multifocal manner inside the swelling ingredient, wherein the said swelling-restricting ingredient is dispersed, and their mechanism of action can vary from common particle-reinforcement to a specific interaction with the swelling ingredient, such as a specific or non-specific cross-linking to yield a bulk of reinforced ingredient. Therefore, in some embodiments according to such systems, the particle size of the swelling-restricting ingredients is insignificant with respect to the same of the swelling ingredient.

In further embodiments, the system also comprises other inactive ingredients, that are used to impart the system improved processability properties or other beneficial traits. A non-limiting, non-exhaustive list of such additives includes lubricants, binders, glidants, fillers, and other ingredients, which can be generally found in solid dosage forms and are described in full in “The Handbook of Pharmaceutical Excipients” and similar editions.

As a further embodiment, the system comprises one or more active drug substances. The preferred drug substance is characterised by preferential absorption through the upper parts of the gastrointestinal tract, namely, stomach, duodenum or jejunum. It can also have an appreciable absorption from ileum. The absorption in colon of such substance is limited relative to the absorption from upper parts of the GI tract. This selective absorption phenomenon is frequently referred to as a “narrow absorption window” (NAW), as already described hereinabove. In another embodiment, the drug is suitable for deployment in the system of present invention, wherein it is more stable in upper parts of the gastrointestinal tract than in the lower ones, or it is more soluble in the former than in the latter, as although technically this phenomenon cannot be termed as narrow absorption window, the manifestations thereof are similar.

In one embodiment, the active pharmaceutical ingredient of this invention is a therapeutically effective amount of at least one drug, either having preferential absorption from the upper parts of gastrointestinal tract, or having better solubility in the upper parts of gastrointestinal tract, or having better stability in the upper parts of gastrointestinal tract.

The novel gastroretentive delivery system of this invention is suitable for use with a multitude of drugs that can draw a potential therapeutic benefit from a prolonged stay in the upper part of the gastrointestinal tract. Examples of such drugs are levodopa, acyclovir, gabapentin, ranitidine, metoprolol and other active pharmaceutical ingredients (APIs).

In one embodiment, the gastroretentive drug delivery system of this invention comprises at least one active pharmaceutical ingredient or diagnostic selected from levodopa, carbidopa, acyclovir, gabapentin, ranitidine, metoprolol, AZT, didanosine, alpha-methyldopa, baclofen, valacyclovir, nitrofurantoin, ciprofloxacin, amoxicillin, cephalexin, lithium carbonate or citrate, calcium carbonate or citrate, riboflavin, ascorbic acid, folic acid, vitamin E, pravastatin, simvastatin, captopril, benazepril, enalapril, cilazapril, fosinopril, ramipril, albuterol, pirbuterol, furosemide, allopurinol, atenolol, metoprolol, cimetidine, famotidine, bismuth subsalicylate, bismuth subcitrate, misoprostol, 5-fluorouracil, doxorubicin, mitomycin, semustine, cisplatin, etoposide, methotrexate, clarithromycin, amoxycillin, metronidazole, zaleplon, methylnaltrexone, a tetracycline, a drug having a narrow absorption window in the gastrointestinal tract, a drug intended for local treatment of the gastrointestinal tract and a drug which degrades in the colon or in the small intestine.

In another embodiment, the therapeutically effective amount of at least one drug which is gabapentin.

In some embodiments, the system comprises more than one drug. Such combinations are known in the art, and can be exemplified by combinations of an ACE inhibitor and hydrochlorothiazide, antiviral combinations, such as ritonavir-lopinavir, oral contraceptive combinations, hormone-replacement therapies, levodopa-carbidopa, levodopa-benserazide, and even triple-ingredient combinations like levodopa-carbidopa-entocapone. Yet combinations that are ever more complex are not uncommon in the cough-and-cold over-the-counter preparations, which can comprise four or even five active entities.

The drug or active pharmaceutical ingredient according to the invention may be any drug suitable for preventing, alleviating, treating or curing a disease or disorder, which is absorbed from the gastrointestinal tract.

The drug or active pharmaceutical ingredient may be selected from drugs having a narrow absorption window in the gastrointestinal tract, as described above. Examples of drugs having a narrow absorption window in the gastrointestinal tract are therapeutic nucleic acid sequences or derivatives, amino acid sequences or derivatives, peptidomimetic drugs, antibiotic agents, therapeutic ions, vitamins, bronchodilators, anti-hypertensive agents, diuretic agents, anti-gout agents, anti-hyperlipidemic agents or ACE inhibitors.

Therapeutic nucleic acid derivatives are, for example, acyclovir, AZT or didanosine. Examples of therapeutic amino acid sequences or their derivatives are gabapentin, levodopa, .alpha.-methyldopa, baclofen or valacyclovir. Examples of antibiotic agents having a narrow absorption window are nitrofurantoin, ciprofloxacin or beta-lactam antibiotic agents such as amoxycillin or cephalexin. Examples of therapeutic ions are lithium carbonate or citrate, calcium carbonate or citrate. Examples of vitamins are riboflavin, ascorbic acid, folic acid or vitamin E. The anti-hyperlipidemic agent may be pravastatin. Examples of ACE inhibitors are captopril, benazepril, enalapril, cilazapril, fosinopril or ramipril. Examples of bronchodilators are albuterol or pirbuterol. Other examples of drugs having a narrow absorption window in the gastrointestinal tract are furosemide, allopurinol or atenolol.

In addition to drugs having a narrow absorption window in the gastrointestinal tract, the delivery system of the invention may comprise a drug for local treatment of the gastrointestinal tract. These may be used, for example, in the treatment of neoplasms of the stomach, such as adenocarcinoma of the stomach or gastric lymphoma.

Examples of drugs for the local treatment of the gastrointestinal tract are anti-tumor agents, histamine (H2) blockers, bismuth salts, synthetic prostaglandins or antibiotic agents. H2 blockers may be cimetidine, famotidine or ranitidine. Bismuth salts may be bismuth subsalicylate or bismuth subcitrate. An example of a synthetic prostaglandin is misoprostol. The anti-tumor drug may be 5-fluorouracil, doxorubicin, mitomycin, semustine, cisplatin, etoposide or methotrexate. Suitable antibiotic agents may be clarithromycin, amoxycillin, metronidazole or a tetracycline.

In addition to the above drugs, which have a narrow absorption window in the gastrointestinal tract or which are intended to local treatment of the gastrointestinal tract, the delivery device of the invention may contain as the active pharmaceutical ingredient a drug which degrades in the colon, for example, metoprolol, or in the small intestine, for example, simvastatin. Any agent having a therapeutic effect in the gastrointestinal tract, or which has a narrow absorption window in the gastrointestinal tract or which preferentially degrades in the lower parts of gastrointestinal tract, other than the aforementioned agents, may be delivered by the device of the invention. Such agents are well known to a person skilled in the art and may be delivered alone or in combination with other suitable therapeutic agents.

One of the preferred drugs is gabapentin.

Therefore, one of the preferred embodiments of the present invention is a method of treatment of patients in need of a drug that is more readily absorbed in the upper part of the gastro-intestinal tract with one or more drugs formulated with the novel gastroretentive delivery system of this invention, which enables a prolonged gastric retention period.

In preferred embodiments, the system of the present invention comprises a tablet. The tablet is produced using professional pharmaceutical equipment for processing of powders. The powders are processed and compressed into the said tablet using a variety of punch-and-die sets, to yield tablets having various forms, sizes and degrees of convexity, as needed for each specific application of the present invention. Such shapes comprise, but are not limited to, round tablets, capsule-shape tablets, known also as caplets, and every possible variation known in the art (octagonal and other polygonal forms, oval, cubic, spherical, etc). Other variations comprise but are not limited to biconvex tablets, flat tablets, lenticular tablets and other vertical-axis modified shaped tablets.

In preferred embodiments the tablet has such shape as to enable easy swallowing, as known in the art.

In another preferred embodiment the tablet has such shape as not to pass easily through the open pyloric valve of a stomach.

In another preferred embodiment the tablet has a biconvex elongated shape, like a caplet.

In another preferred embodiment, the tablet is biconvex and its projection on a plane is an elongated polygon, preferably an elongated octagon.

Sometimes, it is advantageous to produce tablets comprising multiple layers. The purpose for such preparations can be the release at different rates from different compartments, release of two or more drugs at different rates, chemical or physical incompatibility of one or more ingredients in the system, or other causes, as known to the versed in the art. Therefore, in some embodiments, according to the present invention, the tablet comprises two or more layers, wherein the composition of each of the said layers is different from one other. The role of these layers in relation to the present invention is exemplified but not limited to the immediate or modified release compartments, release of two or more drugs at different rates, release-modifying ingredient, such as limiting the diffusion from one or more sides, gastric emptying delaying ingredients, such as bioadhesive layers, flotation enhancement, erosion-prevention and the like.

In some embodiments, the tablet is produced by powder compression using machinery as known in the art. The powders can be alternatively pre-processed jointly or in separate groups, to produce the desired particle size, particle size distribution, flow properties and other important parameters of powder tabletation. In preferred embodiments the pre-processing method is granulation. Granulation process can be exemplified experimentally at the laboratory scale by a mortar and pestle granulation. The ingredients, comprising gas-generating tandem, gas-entrapping hydrogel and swelling ingredient, being also the precursor of swelling-restricting ingredient, are inserted into the mortar and mixed with a pestle and a spatula for time periods from 5 to 20 minutes, thereupon liquid ingredients are added dropwise, and thoroughly mixed for another 5-15 minutes. The obtained mass is dried, mixing the drying mass from time to time. The dried granulated mass is ground using mortar and pestle, and the ingredient is passed through a sieve. The finer particles are retained while the cruder ones are reintroduced into the mortar and grinding is repeated until the entire ingredient is eventually ground. Blending is performed in a suitable vessel, comprising the granulated powders and other ingredients to be blended, like disintegrants, glidants, desiccants or lubricants, which is inverted repeatedly for 15 to 30 minutes to yield homogeneous powders blend, or for 0.5 to 2 minutes, in case of lubricants. Tablets are compressed using an IR tablet press, equipped with a tailor-made punch-die system of a specific shape. Accurately weighed blended powder is inserted into the die and tablets are compressed with the punches. The compression force ranges between 0.5 to 10 tons, depending on other parameters and needs.

In some embodiments, more than one granulation step is performed. In this case the granulated powders from all the granulation steps are blended together as one. In other embodiments, more than one blending step is performed, as the ingredients can be pre-mixed with one another to yield separate blends, which can further be combined into final blend.

While in some embodiments the tablet does not comprise a coating, in yet other embodiments the tablet can comprise a coating, wherein said coating is a rapidly disintegrating coating in the gastric medium. Examples of such coatings are moisture insulating coatings, taste-masking coatings, and aesthetical coatings, as known to those skilled in the art. In these embodiments the coating is applied using coating equipment, such as perforated drum coater. The tablets are pre-heated to process temperature and the coating solution or dispersion in sprayed thereover, controlling the input air temperature and flow and monitoring exhaust air temperature. The coated tablets are thereupon optionally cured as known in the art, and dried in the drum at relevant process temperature and other parameters.

In preferred embodiments, the system of the present invention is advantageously utilised in treatment of various pathological conditions, like every drug-delivery system. These conditions include gastrointestinal (GI) pathological conditions, as well as non-gastrointestinal pathologies. In preferred embodiments the gastrointestinal pathological conditions are treated due to the functionality of the system of present invention, capable of prolonged residence in the upper part of the GI tract. This functionality enables co-localisation of the drug delivery system with the site of action of the drugs, intended for treatment of a GI pathological condition, thereby enabling better efficacy. In further preferred embodiments the GI pathological condition includes disorders, whereby the site of action of the drugs, intended for treatment thereof is the GI tract itself, both GI lumen and the wall. Non-limiting list of examples of such conditions are microfloral misbalance, pseudomembranous colitis, fungal overgrowth of the GI tract, gastritis, colitis, viral or bacterial gastroenteritis, gastric ulcer, gastrooesophageal reflux disease, gastric cancer, irritable bowel syndrome (IBS), GI bleeding, opioid-treatment associated constipation, and other conditions whereby the deficiency or hypersecretion of GI hormones, enzymes, co-factors and such is present.

In further preferred embodiments, the system of the present invention is advantageously utilised in treatment of non-gastrointestinal pathologies, responsive to pharmacotherapy with drugs, having narrow absorption window (NAW). These pathologies are exemplified by the following non-exhaustive non-limiting list: central nervous systems (CNS) disorders, such as Parkinson's disease, Alzheimer's disease symptoms, neuropathic pain, epilepsy, depression, insomnia, psychiatric disorders and others; infectious diseases, including viral infections, such as herpes infections, hepatitis infections and AIDS; metabolic diseases, such as diabetes, dislipidaemia and others; endocrine disorders, including reproductive disorders; cardiovascular pathologies, such as hypertension, congestive heart failure, coagulation disorders and others; renal disorders, such as renal failure, pyelonephritis and others; musculoskeletal system disorders, such as osteoporosis, myasthenia gravis and others; pulmonary disorders, such as pulmonary hypertension; benign and malignant cancer; autoimmune diseases; and other indications whereby administration of drugs, suitable to the system of present invention, is advantageous to treatment of pathological conditions in patients.

In one embodiment of this invention, there is provided a process for the preparation of the gastroretentive controlled-release drug delivery system according of this invention, comprising

a. Mixing at least one ingredient, capable of releasing gas upon contact with gastric fluids, at least one ingredient capable of unrestricted swelling un gastric fluid, at least one ingredient capable of limiting the unrestricted swelling of the said swelling ingredient, and at least one therapeutically active drug, or any combination of ingredients, capable of performing more than one of any of the functions above, to produce an essentially homogeneous blend.
b. Treating the blend with isopropanol, or with an alternative granulation liquid, and mixing together to obtain a homogeneous granulation
c. Drying the resultant granulate to produce a dry granulate
d. Grinding the granulate to produce ground granulate
e. Adding the remainder of the ingredients
f. Compressing tablets of the selected shape, preferably biconvex elongated polygonal shape, comprising one or more layers
g. Optionally coating the resultant tablet with a coating
h. Optionally subjecting to curing by heat at any stage of the process
or any other combination of the above process stages

The gastroretentive drug delivery system of this invention may be used for the treatment of a patient in need thereof.

In one embodiment, there is provided a method of treatment of a patient in need thereof with a gastroretentive drug delivery system of this invention.

In another embodiment, there is provided a method of treatment of a patient or preventing a pathological condition or alleviating of symptoms in patient, suffering from a pathologic condition, a gastrointestinal pathologic condition, such as central nervous systems (CNS) disorders, such as Parkinson's disease, Alzheimer's disease symptoms, neuropathic pain, epilepsy, depression, insomnia, psychiatric disorders and others; infectious diseases, including viral infections, such as herpes infections, hepatitis infections and AIDS; metabolic diseases, such as diabetes, dislipidaemia and others; endocrine disorders, including reproductive disorders; cardiovascular pathologies, such as hypertension, congestive heart failure, coagulation disorders and others; renal disorders, such as renal failure, pyelonephritis and others; musculoskeletal system disorders, such as osteoporosis, myasthenia gravis and others; pulmonary disorders, such as pulmonary hypertension; benign and malignant cancer; autoimmune diseases; microfloral misbalance; pseudomembranous colitis; fungal overgrowth of the GI tract; gastritis; colitis; viral or bacterial gastroenteritis; gastric ulcer; gastrooesophageal reflux disease; gastric cancer; irritable bowel syndrome (IBS); GI bleeding; opioid-treatment associated constipation; and other conditions whereby the deficiency or hypersecretion of GI hormones, enzymes, co-factors and such is present, and other indications whereby administration of drugs, suitable to the system of present invention, is advantageous to treatment of pathological conditions in patients, by administration of the gastroretentive drug delivery system of this invention.

All the methods herein described are well known to those versed in the art, and are subject to suitable changes and adaptations to specific needs of the ingredients and processes. The methods described above can also be carried out in larger-scale equipment, such as industrial granulators, fluid-bed coaters, multipunch rotary tablet presses, blenders, mills and others, known to those well versed in the art. The examples hereinbelow included are given for illustrative purposes only, and should not be deemed as limiting in any respect.

Examples 1-4 below describe the preparation of gastroretentive tablets of this invention without APIs, in order to illustrate the various processes and compositions. APIs in pharmaceutically effective amounts may be added to all the formulations exemplified below, for example as exemplified in Example 5.

It is appreciated that certain features of the invention which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

### EXAMPLES

### Materials

All materials were obtained from local suppliers as follows: albumin from chicken egg, Tween 80 (polysorbate), sodium lauryl sulphate and tannic acid were obtained from Sigma-Aldrich Israel, citric acid, sodium bicarbonate, glutaraldehyde 25% solution and sodium carbonate were obtained from Merck-Israel, povidone of various grades were obtained as free samples from BASF, Eudragit™ polymers were obtained from Degussa, now Evonik, representatives in Israel, glycerine was obtained from Frutarom, Israel. Various excipients and gabapentin were generously donated by Trima, Israel. Solvents and reagents were supplied from the Hebrew University warehouse and comprise several manufacturers.

### Methods

Most of the buffers preparation methods were carried out along the lines described in the United States Pharmacopoeia, technical solutions part. Swelling tests were conducted on an orbit shaker at 37° C., in ca 80 mL of a test buffer. Dissolution tests were conducted according to the USP, using a Vankel dissolution testing apparatus type 2 (USP), equipped with an automatic sampling station, using 900 mL of test medium, at 50 rpm.

The degree of swelling was determined by the immersion of a specimen into the tested liquid and dimensional measurement. This simplest and most straightforward procedure yielded coherent results. Paper towels were used for blotting the specimens to remove excess liquid that was not part of the swollen system. Various alternative methods advise applying pressure to determine the free water inside the system, but this makes the method destructive, and therefore no pressure was applied.

The same method allowed evaluating flotation through simple visual inspection. The vessels comprising the swelling specimens were evaluated visually for floating at initial time and at each swelling kinetics determination point. The true flotation was differentiated from false positive results that could be caused by gas bubbles attached to the surface of the tablet and not entrapped in the interior; for this purpose each buoyant specimen was sunk manually with a spatula, and re-emersion from the bulk of the solution was observed. Positive results were recorded if re-emersion was instantaneous.

Mechanical measurements were necessary to distinguish and quantitate the mechanical properties of the investigated system. The highlight of the project is the maintenance of mechanical strength or de novo development thereof in a swollen tablet. Aluminium probe, 2-mm diameter, was used in penetration tests. An alternative testing with 20-mm diameter probe was performed, designated compression testing, as the whole tablet was deformed. The machine speeds were optimised for best determination of the sought parameters, such as maximal load or load at yield point, which was used to differentiate weak tablets, and the Young's modulus, which was used to discern harder tablets from weaker ones.

Granulation was carried out in a mortar and pestle. The ingredients were inserted into the mortar and mixed with a pestle and a spatula for time intervals from 5 to 20 minutes; thereupon liquid ingredients were added dropwise, and thoroughly mixed for another 5-15 minutes. The obtained mass was dried in an oven in a suitable tray, at designated temperature for some time, mixing the drying mass from time to time.

The dried granulated mass was ground using mortar and pestle, and the material was passed through a sieve with designated mesh. The finer particles were retained while the cruder ones were reintroduced into the mortar and grinding was repeated until the entire amount was eventually ground.

Blending was performed in a suitable closed vessel, comprising the powders to be blended, which was inverted repeatedly for 15 to 30 minutes to yield homogeneous powders blend. Some ingredients were passed through a sieve of designated mesh prior to bending. Blending of magnesium stearate was performed in similar way, for 0.5 to 2 minutes.

Tablets were compressed using an IR tablets press, equipped with a tailor-made punch-die system. The shape (for example round, caplet, biconvex caplet) and the size of the compressed tablet varied according to the needs of the experiment. Accurately weighed blended powder was inserted into the die and tablets were compressed with the punches. In some cases, stearic acid was applied onto dies to prevent sticking of the tablet in the punch. The compression force ranged between 0.5 to 10 tons, depending on other parameters.

### Example 1

### Effect of Denaturation of Egg Albumin with Glycerine on Tablet Properties

Composition

| Ingredient name | 1 | | 2 | |
|---|---|---|---|---|
| Ovalbumin | 1.420 | g | 1.420 | g |
| Citric acid | 0.350 | g | 0.350 | g |
| Sodium bicarbonate | 0.350 | g | 0.350 | g |
| Glycerine | 0.21 | g | 0 | g |
| Magnesium stearate | 0.022 | g | 0.022 | g |

### Preparation: Formulation 1

The powders were blended together for about 10 minutes using mortar and pestle, thereupon, glycerine was added as an aqueous solution ca 1:4 w/w, and mixed. The mixture was heated to 95° C. over 1 hour, on a glass tray, closed with a lid, and the heating was continued for 1.5 more hours in an open tray. The granulate was milled using a mesh 30 sieve, and two tablets were prepared, weighing 610 mg each, using a 12-mm diameter round punch.

### Preparation: Formulation 2

The powders were blended together for about 10 minutes, and two tablets were prepared, weighing 610 mg each, using 12-mm diameter round punch.

### Testing:

The tablets were subjected to simulated gastric fluid without pepsin, USP, and their behaviour was documented

### Results:

The tablets of formulation 1 became instantly buoyant and remained so throughout the test period (over 24 hours).

The tablets of formulation 2 disintegrated over 15 minutes under the effervescent action of gas-releasing tandem.

### Example 2

### Isopropanol Denaturation of Egg Albumin

Ingredients

| Ingredient | 1 | |
|---|---|---|
| Ovalbumin | 1.420 | g |
| Citric acid | 0.350 | g |
| Sodium bicarbonate | 0.350 | g |
| Magnesium stearate | 0.022 | g |
| Isopropanol | 0.47 | g |

### Preparation:

The powders were blended together for about 10 minutes using mortar and pestle, thereupon isopropanol was added and mixed. The mixture was left drying overnight in an open tray at ambient temperature. The granulate was milled using a mesh 30 sieve, and two tablets were prepared, weighing 610 mg each, using IR tabletting press, equipped with a 12-mm diameter round punch.

### Testing:

The tablets were subjected to simulated gastric fluid without pepsin, USP, and their behaviour was documented

### Results:

The tablets became buoyant after 10-15 minutes and remained so throughout the test period.

### Example 3

### Influence of the Swelling Ingredients on the Properties of the Tablets

Compositions

| Ingredient | 1 | | 2 | | 3 | |
|---|---|---|---|---|---|---|
| Ovalbumin | 0.80 | g | 1.20 | g | 0.40 | g |
| Methocel K15M | 0.80 | g | 0.40 | g | 1.20 | g |
| Citric acid | 0.22 | g | 0.22 | g | 0.22 | g |
| Sodium Bicarbonate | 0.20 | g | 0.20 | g | 0.20 | g |
| Sodium lauryl sulphate | 0.16 | g | 0.16 | g | 0.16 | g |
| Crosscarmelose | 0.16 | g | 0.16 | g | 0.16 | g |
| Isopropanol | 0.5 | g | 0.5 | g | 0.5 | g |

### Preparation:

The powders were blended together for about 15 minutes using mortar and pestle, thereupon, isopropanol was added and mixed. The mixture was left drying at 80° C. for one hour, thereupon overnight in an open tray at ambient temperature. The granulate was milled using a mesh 30 sieve, and tablets were prepared, weighing 585 mg each, using IR tabletting press, equipped with a 12-mm diameter round punch. Testing:

The tablets were subjected to simulated gastric fluid without pepsin, USP, and their behaviour was documented.

In addition, mechanical properties of the swollen tablets at the end of experiment (48 hours) were measured using “Texture Analyser” apparatus, produced by Stable Microsystems, of the UK, equipped with a flat-head 20 mm diameter aluminium probe for compression tests, at the following testing parameters: pre-test speed: 3.0 mm/sec, test speed: 0.5 mm/sec, post-test: 0.5 mm/sec, trigger force: 1 g maximal load: 4900 g. All the specimens were fully swollen, as verified by a cross-section examination.

Swelling was measured by blotting the specimens versus a paper towel and performing gravimetric determination on analytical balances, and dimensional measurement using a micrometer.

### Results:

The tablets became buoyant after 5 minutes and remained so throughout the test period.

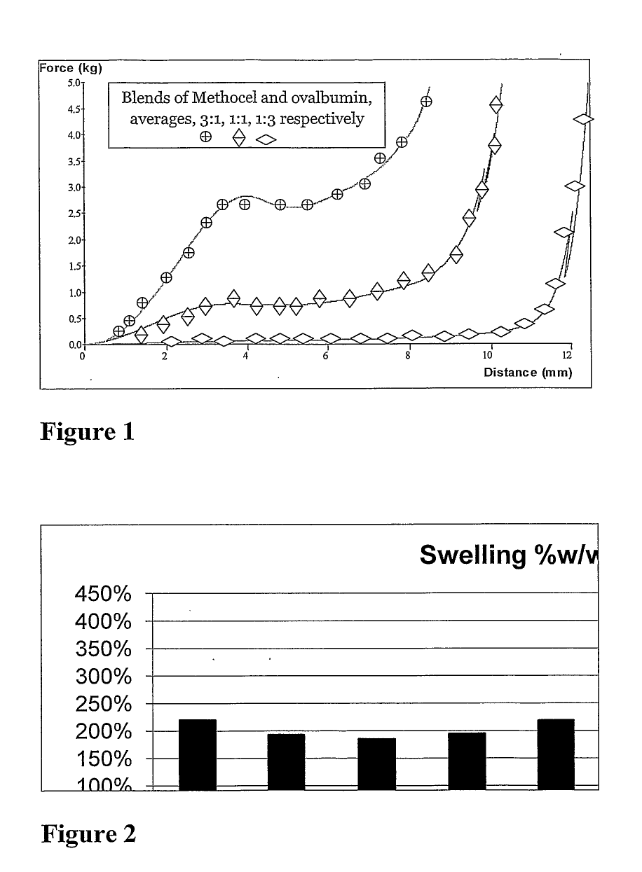

Mechanical properties: the system comprising the most Methocel K15M (experiment 3) yielded the lowest values, followed by the 1:1 system (experiment 1), and 3:1 system (experiment 2) yielding the hardest tablet. The stress-strain graphs from the mechanical testing of the experiment are shown below (FIG. 1).

The swelling of the tablets ranges from 310% (exp. 2) to 540% (exp. 3), which is in accord with prior art.

### Example 4

### Buoyancy, Swelling and Mechanical Properties

### Composition

| Ingredient | 1 | |
|---|---|---|
| Ovalbumin | 4.00 | g |
| Citric acid | 0.55 | g |
| Sodium bicarbonate | 0.50 | g |
| Sodium lauryl sulphate | 0.40 | g |
| Crosscarmelose | 0.40 | g |
| Isopropanol | 1.0 | g |

### Preparation:

The powders were blended together for about 15 minutes using mortar and pestle, thereupon, isopropanol was added and mixed. The mixture was left drying at 80° C. for half-hour. The granulate was milled using mesh 30 sieve, and tablets were prepared, weighing 585 mg each, using IR tabletting press, equipped with a 12-mm diameter round punch.

### Testing:

The tablets were subjected to different buffer solutions, and their behaviour was documented.

Swelling was measured by blotting the specimens versus a paper towel and performing gravimetric determination on analytical balances, and dimensional measurement using a micrometer.

Mechanical testing was performed as in the example above.

### Results:

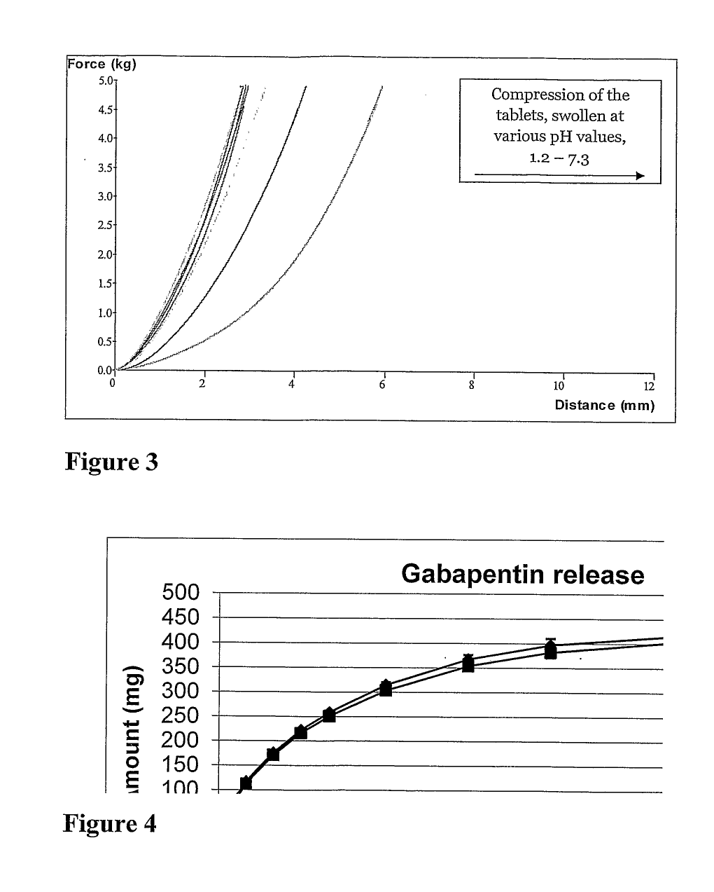

The tablets became buoyant after 5 minutes and remained so throughout the test period. The results of the testing are shown below. FIG. 2 shows the final swelling of the tablet, whereas the FIG. 3 shows the change in mechanical properties as function of pH. It can be seen that the final swelling is increasing as function of pH, climaxing in double-distilled water (DDW). It is noteworthy that in the simulated gastric fluid without pepsin, USP, (SGF), the swelling is higher than at slightly lower pH values. This can be attributed to distancing from isoelectric point, which coincides with the values 3.0-4.0. Mechanical properties follow the opposite pattern, decreasing their value at increasing pH, which is in accord to prior art.

### Example 5

### Gabapentin Tablets

Composition

| Ingredient | 1 | |
|---|---|---|
| Gabapentin | 2.80 | g |
| Ovalbumin | 4.45 | g |
| Citric acid | 0.61 | g |
| Sodium bicarbonate | 0.56 | g |
| Sodium lauryl sulphate | 0.45 | g |
| Crosscarmelose | 0.45 | g |
| Isopropanol | 1.2 | g |
| Magnesium stearate | 0.14 | g |

### Preparation:

### Granulate 1

Gabapentin was wetted with sufficient amount of water to produce a wet mass. The mass was dried at 60° C. for 2 hours in an open tray and milled to pass through mesh-30 sieve.

### Granulate 2

All the powders, apart from gabapentin, were mixed in a mortar with pestle for 15 minutes, and isopropanol was added. The wet granulate was dried at 80° C. for half-hour and milled to pass through a mesh-30 sieve.

### Blending and Tabletting

Both granulates were mixed gently together for 15 minutes, and magnesium stearate was added and mixed for another 2 minutes. The blend was compressed into tablets of caplet shape, weighing 1330 mg each.

### Testing:

Dissolution testing was performed in SGF without pepsin and in acetate buffer at pH 4.0, USP, at 37° C., using Vankel dissolution tester, equipped with pedals (USP type 2 apparatus), in 900 mL of the medium, sampling at 1,2,4,6,8,12,18,24,36 and 48 h.

### Results:

The release profiles are shown in FIG. 4.

What is claimed is:

1. A gastroretentive drug delivery system comprising a tablet suitable for swallowing by a patient, which tablet comprises a therapeutically effective amount of at least one active pharmaceutical ingredient or diagnostic, an inactive ingredient capable of releasing gas upon contact with gastric fluids or a tandem of gas-generating ingredients or an ingredient producing a low-density phase, an inactive ingredient capable of unrestricted swelling in gastric fluid, an inactive ingredient capable of limiting the unrestricted swelling of the said swelling ingredient and an inactive hardening ingredient which allows for the formation of mechanical strength or retention thereof by any mechanism, wherein said inactive ingredients are capable of performing more than one of the functions above.

2. The gastroretentive drug delivery system of claim 1, wherein the unrestricted swelling ingredient is egg albumin or the swelling-restricting ingredient is denaturated egg albumin or both.

3. A gastroretentive drug delivery system in the form of tablet capable of retention in a mammal stomach, wherein said system comprises at least three different gastroretentive mechanisms, complementing one another a. flotation b. swelling and c. hardening or retention of the initial hardness in the swollen state.

4. The gastroretentive drug delivery system of claim 4, wherein the said three different mechanisms are: a. flotation b. swelling and c. hardening or retention of the initial hardness in the swollen state, acting in synergy.

5. The gastroretentive drug delivery system of claim 4, wherein the said three different mechanisms are activated in a specific order: a. rapid onset of floating, within 5 to 15 minutes; b. gradual swelling to final dimensions, not less than within 30 to 60 minutes; c. development or retention of mechanical strength in the swollen state.

6. The gastroretentive drug delivery system of claim 1, wherein the said swelling-restricting ingredient is a small molecule.

7. The gastroretentive drug delivery system of claim 6, wherein the small molecule is chosen from tannic acid, glutaraldehyde and formaldehyde.

8. The gastroretentive drug delivery system according to claim 1, wherein the system becomes buoyant 5 to 30 minutes upon contact with gastric fluid.

9. The gastroretentive drug delivery system of claim 1, wherein the system swells to final dimensions not earlier than within 30 to 60 minutes.

10. The gastroretentive drug delivery system of claim 1, wherein the system, swollen upon the contact with gastric fluid, possesses substantially hard mechanical characteristics, capable of resilience to compressive forces of the stomach.

11. The gastroretentive drug delivery system of claim 1, wherein all three said mechanisms occur during the same performance run.

12. The gastroretentive drug delivery system of claim 1, wherein the system becomes buoyant upon contact with gastric fluid, swells to final dimensions and is hard in the swollen state, in the order of action specified in claim 5.

13. The gastroretentive drug delivery system of claim 1, wherein the said gas releasing ingredient is a physical mixture of gas-releasing basic salt and an organic or inorganic acid.

14. The gastroretentive drug delivery system of claim 13, wherein the gas-releasing mixture comprises a bicarbonate.

15. The gastroretentive drug delivery system of claim 13, wherein the gas-releasing mixture comprises citric acid, tartaric acid, or a mixture thereof.

16. The gastroretentive drug delivery system of claim 13, wherein the gas-releasing mixture further comprises a polymer.

17. The gastroretentive drug delivery system of claim 16, wherein the polymer is a hydrogel, a hydrophobic polymer, or a mixture thereof.

18. The gastroretentive drug delivery system of claim 16, wherein the polymer further comprises a plasticizer.

19. The gastroretentive drug delivery system of claim 1, wherein the said swelling ingredient is a hydro gel, a protein, a water-soluble resin, a gum or an insoluble swelling ingredient.

20. The gastroretentive drug delivery system according to claim 19, wherein the swelling ingredient is chosen from hypromelose, methyl cellulose, hydroxypropyl cellulose, hydroxyethyl cellulose, carboxymethyl cellulose, guar gum, xanthan gum, locust bean gum, tragacanth gum, carrageenan, pectin, dextrins, egg albumin, bovine serum albumin, casein, gelatine, zein, polyvinyl alcohol, graft polyvinyl alcohol-polyethylene glycol, polyethylene oxides, microcrystalline cellulose, cross-linked carboxymethyl cellulose, cross-linked polyvinyl pyrrolidone, polacrylin, or a starch.

21. The gastroretentive drug delivery system of claim 1, wherein the at least one ingredient capable of limiting the unrestricted swelling of the said swelling ingredient is a polymer.

22. The gastroretentive drug delivery system of claim 21, wherein the polymer is an elastic polymer, capable of substantial elongation under stress.

23. The gastroretentive drug delivery system, according to claim 22, wherein the said elastic polymer comprises a plasticizer.

24. The gastroretentive drug delivery system of claim 21, wherein the polymer is a protein, wherein the protein is chosen from egg albumin, casein, gelatine and its derivatives, collagen; said proteins being optionally partially or fully denaturated, or partially or fully cross-linked.

25. The gastroretentive drug delivery system of claim 21, wherein the polymer is a synthetic polymer, wherein the polymer is chosen from polymethacrylates, ethyl cellulose, hypromelose phthalate, hydroxypropyl methyl cellulose acetate succinate, cellulose acetate phthalate, cellulose acetate, cellulose acetate butyrate and other polymers.

26. The gastroretentive drug delivery system, according to claim 21, wherein the polymer further comprises a plasticizer, said suitable plasticiser being selected from polyethylene glycols, poloxamer block copolymers, citrates, such as triethyl citrate or tributyl citrate, phthalates, such as diethyl phthalate, dibutyl sebacate, mineral oil, triglycerides, fatty acids, fatty acids alcohols, such as cetyl alcohol, and others.

27. The gastroretentive drug delivery system of claim 21, wherein the polymer is cross-linked egg albumin.

28. The gastroretentive drug delivery system of claim 27, wherein the cross-linked egg albumin is produced through controlled denaturation of native egg albumin powder.

29. The gastroretentive drug delivery system, of claim 28, wherein the controlled denaturation of native egg albumin powder is instigated by an organic solvent.

30. The gastroretentive drug delivery system of claim 29, wherein the organic solvent is isopropanol.

31. The gastroretentive drug delivery system of claim 30, wherein organic solvent is added during the granulation step.

32. The gastroretentive drug delivery system of claim 31 wherein said system further comprises a surface-active material (SAM).

33. The gastroretentive drug delivery system of claim 32, wherein the surface-active material (SAM) is selected from a cationic SAM, an anionic SAM, a zwitterionic SAM or a non-ionic SAM.

34. The gastroretentive drug delivery system of claim 32, wherein the said surface-active material is selected from polysorbates, poloxamers, sodium alkyl sulphonates and other related ingredients.

35. The gastroretentive drug delivery system of claim 1, wherein the said active pharmaceutical ingredient is a therapeutically effective amount of at least one drug, either having preferential absorption from the upper parts of gastrointestinal tract, or having better solubility in the upper parts of gastrointestinal tract, or having better stability in the upper parts of gastrointestinal tract.

36. The gastroretentive drug delivery system of claim 1 wherein the at least one active pharmaceutical ingredient or diagnostic is selected from levodopa, carbidopa, acyclovir, gabapentin, ranitidine, metoprolol, AZT, didanosine, alpha-methyldopa, baclofen, valacyclovir, nitrofurantoin, ciprofloxacin, amoxicillin, cephalexin, lithium carbonate or citrate, calcium carbonate or citrate, riboflavin, ascorbic acid, folic acid, vitamin E, pravastatin, simvastatin, captopril, benazepril, enalapril, cilazapril, fosinopril, ramipril, albuterol, pirbuterol, furosemide, allopurinol, atenolol, metoprolol, cimetidine, famotidine, bismuth subsalicylate, bismuth subcitrate, misoprostol, 5-fluorouracil, doxorubicin, mitomycin, semustine, cisplatin, etoposide, methotrexate, clarithromycin, amoxycillin, metronidazole, zaleplon, methylnaltrexone, a tetracycline, a drug having a narrow absorption window in the gastrointestinal tract, a drug intended for local treatment of the gastrointestinal tract and a drug which degrades in the colon or in the small intestine.

37. The gastroretentive drug delivery system of claim 1, wherein the active pharmaceutical ingredient is a therapeutically effective amount of at least one drug which is gabapentin.

38. A process for the preparation of a gastroretentive controlled-release drug delivery system according to the claim 1, comprising
a. mixing at least one ingredient, capable of releasing gas upon contact with gastric fluids, at least one ingredient capable of unrestricted swelling un gastric fluid, at least one ingredient capable of limiting the unrestricted swelling of the said swelling ingredient, and at least one therapeutically active drug, or any combination of ingredients, capable of performing more than one of any of the functions above, to produce an essentially homogeneous blend;
b. treating the blend with isopropanol, as in claim 32, or with an alternative granulation liquid, and mixing together to obtain a homogeneous granulation;
c. drying the resultant granulate to produce a dry granulate;
d. grinding the granulate to produce ground granulate;
e. adding the remainder of the ingredients;
f. compressing tablets of the selected shape, preferably biconvex elongated polygonal shape, comprising one or more layers;
g. optionally coating the resultant tablet with a coating;
h. optionally subjecting to curing by heat at any stage of the process;
or any other combination of the above process stages.

39. A method of treatment of a patient in need thereof with a gastroretentive drug delivery system of claim 1.

40. A method of treatment of a patient or preventing a pathological condition or alleviating of symptoms in patient, suffering from a pathologic condition, a gastrointestinal pathologic condition, such as central nervous systems (CNS) disorders, such as Parkinson's disease, Alzheimer's disease symptoms, neuropathic pain, epilepsy, depression, insomnia, psychiatric disorders and others; infectious diseases, including viral infections, such as herpes infections, hepatitis infections and AIDS; metabolic diseases, such as diabetes, dislipidaemia and others; endocrine disorders, including reproductive disorders; cardiovascular pathologies, such as hypertension, congestive heart failure, coagulation disorders and others; renal disorders, such as renal failure, pyelonephritis and others; musculoskeletal system disorders, such as osteoporosis, myasthenia gravis and others; pulmonary disorders, such as pulmonary hypertension; benign and malignant cancer; autoimmune diseases; microfloral misbalance; pseudomembranous colitis; fungal overgrowth of the GI tract; gastritis; colitis; viral or bacterial gastroenteritis; gastric ulcer; gastrooesophageal reflux disease; gastric cancer; irritable bowel syndrome (IBS); GI bleeding; opioid-treatment associated constipation; and other conditions whereby the deficiency or hypersecretion of GI hormones, enzymes, co-factors and such is present, and other indications whereby administration of drugs, suitable to the system of present invention, is advantageous to treatment of pathological conditions in patients, by administration of the gastroretentive drug delivery system of claim 1.
